(19) United States
(12) Patent Application Publication
(10) Pub. No.: US 2012/0328521 A1
(43) Pub. Date: Dec. 27, 2012

(54) FORMULATIONS SUITABLE FOR PET IMAGING WITH HYDROPHOBIC PET AGENTS

(75) Inventors: Carsten Olbrich, Berlin (DE); Michael Krause, Berlin (DE); Andreas Burkhard, Falkensee (DE)

(73) Assignee: PIRAMAL IMAGING SA, Matran (CH)

(21) Appl. No.: 13/518,197

(22) PCT Filed: Dec. 22, 2010
(86) PCT No.: PCT/EP10/70455

§ 371 Date: Sep. 10, 2012

(30) Foreign Application Priority Data
Dec. 23, 2009 (EP) 09075568.7

(51) Int. Cl. A61K 51/04

(52) U.S. Cl. 424/1.89

(57) ABSTRACT

The invention is directed to formulations of lipophilic Amyloid βeta ligand stilbene based derivatives and more particularly to formulations which are administrable parentally e.g. intravenously wherein the lipophilic Amyloid βeta ligand stilbene based derivative is a ¹⁹F or ¹⁸F-labeled radiopharmaceutical thereof. Further, the invention is directed to a method for sterile filtration of said suitable formulation.

### FIELD OF THE INVENTION

The invention is directed to formulations of lipophilic Amyloid βeta ligand stilbene based derivatives and more particularly to formulations which are administrable parentally e.g. intravenously wherein the lipophilic Amyloid βeta ligand stilbene based derivative is a ¹⁸F-labeled radiopharmaceutical thereof. Further, the invention is directed to a method for sterile filtration of said suitable formulation.

### BACKGROUND

Stilbene derivatives useful for Positron Emission Tomography (PET) imaging of patient are known from WO2003/018070A1 and WO2006/066104A1. Stilbene derivatives are radiolabeled with ¹⁸F radioisotope whereas radiolabeling occurred in organic solution in presence of the stilbene derivative precursor and [¹⁸F]. The stilbene derivative precursor can be in a dry condition and optionally has an inert pharmaceutically acceptable carrier and/or auxiliary substances added thereto and a reducing agent and optionally a chelator. The fluoro-radiolabeled stilbene derivative may contain any additive such as pH controlling agent (e.g. acids, bases, buffers), stabilizers (e.g. ascorbic acid) or isotonizing agents (e.g. sodium chloride).

Usually, PET supply centers produce on demand a hot stock solution comprising the radiopharmaceutical that is injected to the patient along the working day. The hot stock solution must be stable and storable. Until now, there has been little published on formulations suitable for PET-radiopharmaceuticals.

Thus, there is a need for commercially acceptable suitable formulations comprising a PET agent characterized in that the PET agent shows a low water solubility i.e. lipophilic PET agent wherein the PET agent is a Aβ ligand stilbene based derivative useful for PET imaging.

It has been surprisingly found that the radiopharmaceutical formulation is chemically stable and can be stored more than 8 hours and that this formulation allows the sterile filtration using suitable filter material(s) without loss of activity.

It has been found that fluoro radiolabeled stilbene derivatives are solubilized and stabilized by the formulation of present invention. Using this formulation, dilutions needed for adjustment of activity can be made in a wide range of dilution ratios, allowing the precise adjustment for any patient at any given time of the shelf life. It was demonstrated, that this formulation is not only useful for the solubilization of Aβ ligand stilbene based derivatives, but for other hydrophobic PET agents also. It combines good local tolerability with easy applicability within the manufacturing process for the radiolabeled PET tracer.

Sterile filtration step is necessary for providing a sterile parenteral formulation and the like for obtaining a suitable pharmaceutical solution for pharmaceutical use. Unfortunately, a critical loss of fluoro labelled ingredient is in many cases observed. Thus, there is a need for improving the purification steps leading to an increase of the radio-labelling yield.

It has been surprisingly found that the suitable formulation of the present invention is successfully used with a sterile filter reducing adsorption onto a sterile filter of the radiopharmaceutical.

### SUMMARY

The invention is directed to formulations of lipophilic Amyloid βeta ligand stilbene based derivatives and more particularly to formulations which are administrable parentally e.g. intravenously wherein the lipophilic Amyloid βeta ligand stilbene based derivative is a ¹⁸F-labeled radiopharmaceutical thereof. Further, the invention is directed to a method for sterile filtration of said suitable formulation.

### DETAILED DESCRIPTION

The present invention concerns formulations comprising radiopharmaceutical wherein the formulation is suitable for parental administration into mammal.

In a first aspect, the invention is directed to formulations comprising
Lipophilic Amyloid βeta ligand stilbene based derivative,
Alcohol, and
Polyether.

### Lipophilic Amyloid βeta Ligand Stilbene Based Derivative:

The Lipophilic Amyloid βeta ligand stilbene based derivative is preferably a compound of formula I

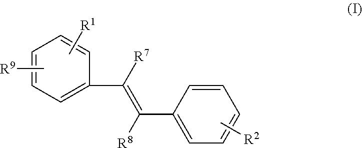

or a suitable salt thereof.
   wherein
   R¹ is selected from the group comprising:
NR³R⁴,
hydroxy,
C₁₋₄ alkoxy,
hydroxy(C₁₋₄)alkyl,
halogen,
cyano,
hydrogen,
nitro,
(C₁-C₄)alkyl,
Halo(C₁-C₄)alkyl, and
Formyl;
   R³ and R⁴ are independently hydrogen, C₁₋₄ alkyl or (CH₂)_{d}R⁵, and d is an integer between 1 and 4;
   R⁹ is selected from the group comprising R⁵, hydrogen, R⁵—(C₁₋₄)alkyl, [R⁵—(C₁₋₄)alkyl]amino, [R⁵—(C₁-C₄)alkyl]alkylamino, and R⁵—(C₁-C₄)alkoxy;
   R² is selected from the group consisting of hydroxyl, C₁₋₄Alkoxy, (C₁-C₄)-alkyloxo Alk(C₁-C₄)oxy, (C₁-C₄)-alkyloxo(C₁-C₄)-alkyloxo(C₁-C₄)alkoxy, (C₁-C₄)-alkyloxo(C₁-C₄)-alkyloxo(C₁-C₄)-alkyloxo(C₁-C₄)alkoxy, carboxy(C₁-C₄) Alkyl, halo(C₁-C₄)alkoxy, halo(C₁-C₄)-alkyloxo(C₁-C₄)alkoxy, halo(C₁-C₄)alkyloxo(C₁-C₄)alkyloxo-alkyloxy, halo(C₁-C₄)alkyloxo(C₁-C₄)alkyloxo(C₁-C₄)alkyloxo(C₁-C₄)alkyloxy, halo(C₁-C₄)alkyl, NR⁶R¹⁰, phenyl(C₁-C₄)alkyl, R⁵—(C₁-C₄)alkoxy, R⁵—(C₁-C₄)alkyloxo(C₁-C₄)alkoxy, R⁵—(C₁-C₄)alkyloxo(C₁-C₄)alkyloxo(C₁-C₄)alkyloxy, R⁵—(C₁-C₄)alkyloxo(C₁-C₄)alkyloxo(C₁-C₄)alkyloxo(C₁-C₄)alkyloxy, R⁵—(C₁-C₄)alkyl;

### R⁵ is ¹⁸F or ¹⁹F;

R⁶ and R¹⁹ are independently selected from the group comprising hydrogen, hydroxy(C₁-C₄)alkyl and C₁-C₄alkyl;
R⁷ and R⁸ are in each instance independently selected from the group comprising halogen, hydrogen, hydroxy, amino, methylamino, dimethylamino, C₁₋₄ alkoxy, C₁₋₄ alkyl, and hydroxy(C₁₋₄)alkyl.

In a preferred embodiment, R¹ is NR³R⁴, wherein R³ and R⁴ are independently hydrogen, or C₁₋₄ alkyl, and R⁹ is hydrogen. More preferably, R¹ is NR³R⁴, wherein R³ and R⁴ are independently hydrogen or C₁ alkyl, and R⁹ is hydrogen.

In a preferred embodiment, R² is R⁵—(C₁-C₄)alkoxy, R⁵—(C₁-C₄)alkyloxo(C₁-C₄)alkoxy, R⁵—(C₁-C₄)alkyloxo(C₁-C₄)alkyloxo(C₁-C₄)alkyloxy, R⁵ (C₁-C₄)alkyloxo(C₁-C₄)alkyloxo(C₁-C₄)alkyloxo(C₁-C₄)alkyloxy, R⁵—(C₁-C₄)alkyl and preferably alkyloxo is a C₁-C₂ alkyloxo. More preferably, R² is R⁵—C₂-alkoxy, R⁵—C₂-alkyloxoC₂-alkoxy, R⁵—C₂-alkyloxo C₂-alkyloxo C₂-alkyloxy, R⁵—C₂-alkyloxo C₂-alkyloxoC₂alkyloxo C₁alkyloxy, R⁵—C₄-alkyl. Even more preferably, R² is R⁵—C₂-alkyloxo C₂-alkyloxo C₂-alkyloxy.

In a preferred embodiment, R⁷ and R⁸ are in each instance independently selected from the group comprising halogen, hydrogen, hydroxy or amino. More preferably, R⁷ and R⁸ are hydrogen.

In a preferred embodiment, the lipophilic Amyloid βeta ligand stilbene wherein R⁵ if ¹⁸F is administered such that the dose of the radiopharmaceuucal is in the range of 37 MBq (1 mCi) to 740 MBq (20 mCi). In particular, a dose in the range from 150 MBq to 370 MBq will be used.

Invention compounds are present in the formulation at a maximum concentration of 10 μg/mL at RT and preferably is 5 μg/mL at RT.

Preferred lipophilic Amyloid βeta ligand stilbene based derivatives are

### Compound 1:

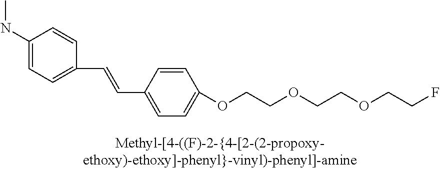

and

### Compound 2:

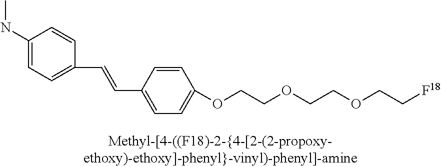

Preferred formulation comprises
Compound 1

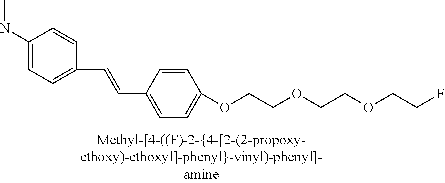

Alcohol, and
Polyether.
   Other preferred formulation comprises
Compound 2

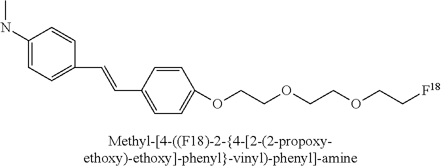

Alcohol, and
Polyether.

Additionally and optionally the formulation of the present invention comprises Ascorbid acid, Sodium dihydrogenphosphate dihydrate, and/or Sodium monohydrogenphosphate dihydrate or any pH adjusting agent known in the art.

### Alcohol:

In a preferred embodiment, the alcohol is present into the formulation in an amount of about 8% v/v to 20% v/v. Preferably, the alcohol is present in an amount of about 10% v/v to 15% v/v, more preferably 15% v/v. The alcohol is an alcohol with a carbon chain length of at least 2, Preferably, the alcohol is a C₂-C₅ alcohol. More preferably, the alcohol is C₂, C₃ or C₄ alcohol. Alcohol is preferably ethanol. The ethanol is a 96% up to 100% ethanol.

### Polyether:

In a preferred embodiment, the polyether is present into the formulation in an amount of about 10% v/v to 25% v/v. Preferably, the polyether is present in an amount of about 8% v/v to 20% v/v more preferably 20% v/v. Polyether is preferably a poly(ethylene glycol) (PEG), such as PEG 300, PEG 400 or PEG 1500.

The formulations of the present invention are pharmaceutical formulations suitable for parental administration into mammals.

A preferred formulation comprises
a compound of formula I

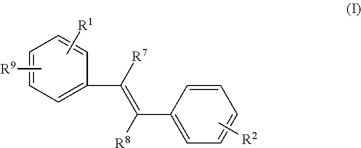

or a suitable salt thereof
wherein
R¹ is NR³R⁴, wherein R³ and R⁴ are independently hydrogen or C₁ alkyl;
R⁹ is hydrogen;
R² is R⁵—C₂-alkyloxo C₂-alkyloxo C₂-alkyloxy;
R⁷ and R⁸ are hydrogen and
R⁵ is ¹⁸F or ¹⁹F;
Ethanol 96% in an amount of about 10% v/v to 15% v/v and
Polyethylenglycol (PEG 400) in an amount of about 8% v/v to 20% v/v.

More preferred formulation comprises
Compound

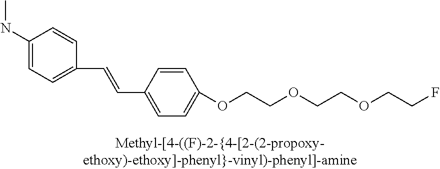

or

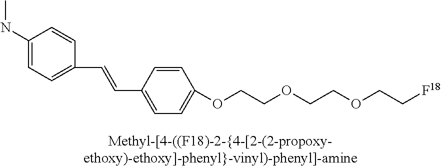

or mixtures thereof
   Ethanol≧96% in an amount of about 15% v/v and
   Polyethylenglycol (PEG 400) in an amount of about 20% v/v.

In a further embodiment, the invention is directed to a formulation comprising
Lipophilic Amyloid βeta ligand stilbene based derivative,
Alcohol,
Polyether and
pH adjusting agent.

Preferably, the formulation comprises
Compound 1

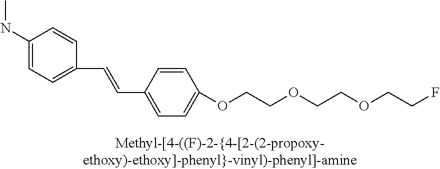

or compound 2

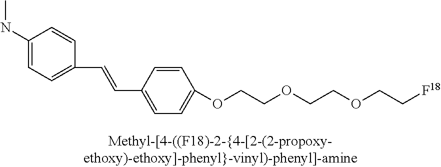

or mixtures thereof
Alcohol,
Polyether, and
pH adjusting agent.

Ascorbid acid and Sodium monohydrogenphosphate dihydrate are pH adjusting agent known in the art.

In a further embodiment, the invention is directed to a formulation comprising
Lipophilic Amyloid βeta ligand stilbene based derivative,
Alcohol,
Polyether,
Ascorbid acid, and
Sodium monohydrogenphosphate dihydrate.

Preferably, the formulation comprises
Compound 1

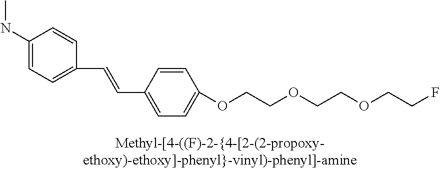

or compound 2

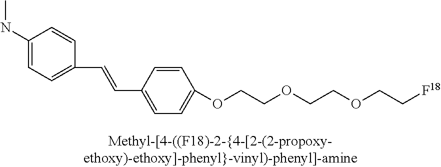

or mixtures thereof
Alcohol,
Polyether,
Ascorbid acid, and
Sodium monohydrogenphosphate dihydrate.

Preferably, the compound 1 or 2 or mixture thereof is present into the formulation in an amount of about 0.0001 to 0.0010% w/v. More preferably, in an amount of about 0.0003 w/v or 0.0005% w/v.

Preferably, the alcohol is present into the formulation in an amount of about 8% v/v to 20% v/v. More preferably, the alcohol is present in an amount of about 10% v/v to 15% v/v, more preferably 15% v/v. The alcohol is an alcohol with a carbon chain length of at least 2, Preferably, the alcohol is a C₂-C₅ alcohol. More preferably, the alcohol is C₂, C₃ or C₄ alcohol. Alcohol is preferably ethanol. The ethanol is a 96% up to 100% ethanol.

Preferably, the polyether is a poly(ethylene glycol) (PEG), such as PEG 300, PEG 400 or PEG 1500. More preferably, the polyether, for example PEG 400, is present into the formulation in an amount of about 10% w/v to 25% w/v. Even more preferably, the polyether, for example PEG 400, is present in an amount of about 8% w/v to 20% w/v more preferably 20% w/v.

Preferably, Ascorbid acid is present into the formulation in an amount of about 0.1% w/v to 2% w/v. More preferably, Ascorbid acid is present in an amount of about 0.1% w/v to 1 w/v. Even more preferably, Ascorbid acid is present in an amount of about 0.1% w/v to 0.5% w/v.

Preferably, Sodium mono-hydrogenphosphate-dihydrate is present into the formulation in an amount of about 0.1% w/v to 2% w/v. More preferably, Sodium mono-hydrogenphosphate-dihydrate is present in an amount of about 0.1% w/v to 1% w/v. Even more preferably, Sodium mono-hydrogenphosphate-dihydrate is present in an amount of about 0.1% w/v to 0.5% w/v.

More preferably, the formulation comprises

Compound 1 or 2 or mixture thereof is about 0.0001 to 0.0010% w/v,
Ethanol 96% (V/V) is about 8% v/v to 20% v/v,
PEG 400 is about 10% v/v to 25% v/v,
Ascorbic acid is about 0.1% w/v to 2% w/v and
Sodium mono-hydrogenphosphate-dihydrate is about 0.1% w/v to 2% w/v.

Even more preferably, the formulation contains

0.0005% w/v of Compound 1 or 2 or mixture thereof

### 15% v/v of Ethanol 96°/0 (V/V),

### 20% w/v of PEG 400,

0.2% w/v of Ascorbic acid
Sodium mono-hydrogenphosphate-dihydrate is 0.25% w/v, and water as a rest.

Preferably, the formulation comprises compound 1.

Preferably, the formulation comprises compound 2.

In a second aspect, the invention is directed to a method for preparing the formulation of the present invention comprising a lipophilic Amyloid βeta ligand stilbene based derivative. Preferably the lipophilic Amyloid βeta ligand stilbene based derivative is a compound of formula I as disclosed above.

The method comprises the steps of
Solubilisation lipophilic Amyloid βeta ligand stilbene based derivative in alcohol and
Adding the alcohol solution of first step into polyether.

Embodiment disclosed above for lipophilic Amyloid βeta ligand stilbene based derivative, alcohol and polyether are included herein.

Preferably, the method comprises the steps of
Solubilisation of compound 1 or 2 or mixture thereof in alcohol and
Adding the alcohol solution of first step into polyether.

Additionally and optionally, a pH adjusting agent is added to the obtained formulation.

In a third aspect, the invention is directed to a method for sterile filtration of the formulation of the present invention comprising a lipophilic Amyloid βeta ligand stilbene based derivative. Preferably the lipophilic Amyloid βeta ligand stilbene based derivative is a compound of formula I as disclosed above.

It was surprisingly found that the adsorption onto sterile filter is strongly decreased when the formulation of the present invention is used. The sterile filter can be standard sterile filter used for radiotracer filtration. Such sterile filters are well known in the art.

The method for sterile filtration of the formulation of the present invention comprises the step of giving the formulation of the present invention onto a sterile filter.

The lipophilic Amyloid βeta ligand stilbene based derivative of formula is a hydrophobic substance and the formulation allows the dissolution of the substance at the required doses. It's well known and acknowledged that hydrophobic filters have an affinity for hydrophobic substances. The use of solvents/co-solvents does reduce adsorption of hydrophobic substances onto hydrophobic filters. Additionally, it was found, that the formulation of the present invention prevents this adsorption and allows a high yield sterile filtration.

Preferably, the method for sterile filtration of the formulation of the present invention comprises the step of giving the formulation of the present invention onto polytetrafluoroethylene (PTFE) sterile filter e.g Sartorius Minisart 0.2 μm, Order number 16596 or Polyvinylidene Fluoride (PVDF) sterile filter e.g. Millipore Millex 0.2 μm SLGV013SL.

More preferably, the hydrophobic filter is polytetrafluoroethylene (PTFE) sterile filter.

Optionally, the sterile filtration method is preceded by the preparation of the formulation of the present invention.

Embodiment disclosed above for lipophilic Amyloid βeta ligand stilbene based derivative, alcohol and polyether are included herein.

In a fourth aspect, the invention is directed to the use of the formulation of the present invention for the manufacture of a suitable PET imaging agent for parenteral administration to mammals.

In a fifth aspect, the invention is directed to the use of the formulation of the present invention for the manufacture of a suitable radiotherapy medicament for parenteral administration to mammal.

In a sixth aspect, the invention is directed to
A device for the preparation of the invention formulation comprising a radiotracer obtained though an automated device for radiopharmaceutical use,
A method for the preparation of the invention formulation comprising a radiotracer obtained though an automated device for radiopharmaceutical use.

Inventors have found a method for obtaining an invention formulation that can be easily integrated into the radiopharmaceutical processes conducted onto automated devices.

The method for the preparation of the invention formulation comprising a radiotracer obtained though an automated device for radiopharmaceutical use comprises the steps:
Obtaining a radiotracer,
Purification of the radiotracer using a solid-phase-extraction cartridges or column wherein the radiotracer is eluted with alcohol,
Adding the alcohol eluat into polyether for obtaining the invention formulation and
Sterile filtration of the formulation.

The radiotracer is a lipophilic Amyloid βeta ligand stilbene based derivative such as compound 2. Alcohol and polyether are as defined above.

### DEFINITION

The terms used in the present invention are defined below but are not limiting the invention scope.

Suitable salts of the compounds according to the invention include salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disul-phonic acid, acetic acid, trifluoroacetic acid, propionic acid, lactic acid, tartaric acid, malic acid, citric acid, fumaric acid, maleic acid and benzoic acid.

Suitable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, diben-zylamine, N-methylmorpholine, arginine, lysine, ethylenediamine and N-methylpiperidine.

Halogen means Chloro, Iodo, Fluoro and Bromo. Preferably, halogen means Iodo or Bromo.

The term “alkyl” as used herein refers to C₁ to C₄ straight or branched alkyl groups, e.g., methyl, ethyl, propyl, isopropyl, n-butyl, or t-butyl. Alkyl groups can be perfluorated or substituted by one to three substituents selected from the group consisting of halogen, hydroxyl or C₁-C₄ alkoxy. More preferably, alkyl is a C₁ to C₂ or C₁ to C₃ alkyl.

The term “alkoxy” as used herein refers to —O—C₁ to C₄ straight or branched alkyl groups.

Polyethers are compounds with more than one ether group. While the term generally refers to polymers like polyethylene glycol and polypropylene glycol, low molecular compounds such as the crown ethers may sometimes be included.

A radiopharmaceutical or radiotracer is a compound suitable for use in medical applications such as nuclear imaging, chemotherapy and the like. Radiopharmaceuticals are generally provided in a pharmaceutically-acceptable carrier.

A suitable formulation is rendered suitable for pharmaceutical use by adjusting the pH, concentration or other physical characteristics of pharmaceutical preparation well known in the art.

Unless otherwise specified, when referring to the compounds of formula the present invention per se as well as to any pharmaceutical composition thereof the present invention includes all of the hydrates, salts, and complexes.

### EXPERIMENTAL DATA

### 1. Compounds 1 (Fluoro-Labeled) and 2 (Fluoro-Radiolabeled)

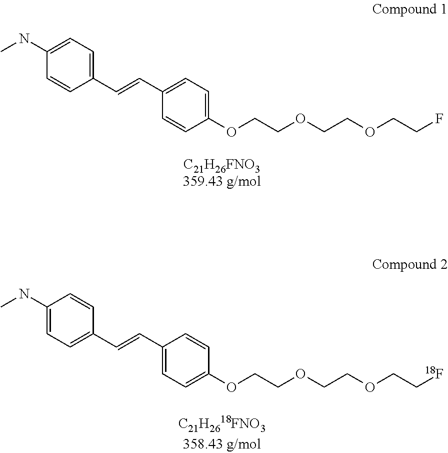

### 2. Formulations Comprising Lipophilic Amyloid βeta Ligand Stilbene Based Derivative, General Procedure

To mimic the manufacturing procedure in the radiopharmacy department the following procedure was developed.

### Formulation I Compound 1:

Ascorbic acid, Sodiumdihydrogenphosphate-dihydrate, di-Sodium hydrogenphosphate-dihydrate were weighed together. Then, PEG and water were added. All ingredients were dissolved by stirring. Finally the Ethanol and Aβ ligand stilbene based derivative compound 1 were added. The preparation is mixed.

| Ingredients | Formulation I | |
|---|---|---|
| Compound 1 | 54 μg HCl (50 μg base, | |
| | compound 1) | |
| Ethanol 96% | 1.2075 | g |
| Polyethylenglycol (PEG 400) | 2 | g |
| Ascorbic acid | 20 | mg |
| Sodium monohydrogenphosphate - | 26.7 | mg |
| dihydrate | | |
| Sodiumdihydrogenphosphate- | 93.6 | mg |
| dihydrate | | |
| Water | ad 10 | g |

| | |
|---|---|
| pH | Around 6.8 |

Since solutions of stilbenes are light sensitive, the solutions were stored under light protection.

Solubility of the active was tested by visual inspection using an illuminated magnifying glass with black background and confirmed by experiments assessing particulate matter using the HIAC Royco, Liquid Particle Counting System, Model 9703.

Using this procedure, maximum solubility, as well as formulation alternatives/different co-solvents, different amounts of Ethanol and different amounts of PEG 400 were assessed.

### Formulation II, Compound 1:

| Ingredients | Formulation II | |
|---|---|---|
| Compound 1 | 55.1 μg HCl | |
| Ethanol 96% | 1.215 | g |
| Polyethylene glycole (PEG 400) | 2 g (ca. 1.8 mL) | |
| Ascorbic acid | 20 | mg |
| Sodium monohydrogenphosphate - | 25 | mg |
| dihydrate | | |
| Water | ad 10 | mL |

| | |
|---|---|
| pH | 6.84 |

Methology is as disclosed above for formulation I.

### 3. Stability of Formulation Comprising HCl Salt of Compound 1 for 8 Hours at Room Temperature

A solution was prepared containing 5.51 μg/mL HCl salt of compound 1 as in formulation I. Assay was analysed after different timepoints. Three individual batches were prepared and analysed for assay and particulate matter. From these solutions samples were taken and analysed by HPLC.

Table 1 indicates the results of stability testing up to 8 hours of 3 individually manufactured batches.

The assay of HCl salt of compound 1 stays within the 95% to 105% interval within the 8 hour observation time and there is no trend of a reduction over time. HCl salt of compound 1 can be considered to be chemically stable in the formulation.

TABLE 1

| time (hours) | batch 01 | batch 02 | batch 03 | mean [%] | SD [%] |
|---|---|---|---|---|---|
| 1 | 100.2 | 98.3 | 96.9 | 98.5 | 1.7 |
| 2 | 101.2 | 98.4 | 97.17 | 98.9 | 2.1 |
| 3 | 101.1 | 98.7 | 96.2 | 98.7 | 2.5 |
| 4 | 101.2 | 98 | 97.4 | 98.9 | 2.0 |
| 5 | 101.5 | 98.6 | 96.6 | 98.9 | 2.5 |
| 6 | 101.5 | 98.7 | 95.7 | 98.6 | 2.9 |
| 7 | 101.7 | 98.5 | 96.5 | 98.9 | 2.6 |
| 8 | 102.7 | 98.7 | 96.5 | 99.3 | 3.1 |

### 4. Particles Formation in Formulation Comprising HCl Salt of Compound 1

The formation of particles was assessed using the HIAC Royco, Liquid Particle Counting System, Model 9703 and in addition to the channels normally inspected (10 μm and 25 μm), also the smaller channels (2 μm and 5 μm) were used to assess the stability of the formulation. The formulation I was sterile filtered and inspected at timepoint 1 hour and 8 hours.

Table 2 indicates the results of particulate matter testing up to 8 hours of 3 individually manufactured batches

Compound 1 remains dissolved and is not precipitating. Since the handling of the solutions was made under normal laboratory conditions, the particle background measured in the sterile filtered solutions have an exogenous nature.

TABLE 2

| | |
|---|---|
| 10 mL | Cumulative Count |

| Particle | Batch | Batch | Batch | Batch | Batch | Batch |
|---|---|---|---|---|---|---|
| Size | 01 | 01 | 02 | 02 | 03 | 03 |
| [μm) | 1 h | 8 h | 1 h | 8 h | 1 h | 8 h |
| 2 | 336 | 218 | 1508 | 1578 | 475 | 575 |
| 5 | 212 | 109 | 507 | 584 | 176 | 239 |
| 10 | 103 | 50 | 213 | 287 | 78 | 131 |
| 15 | 8 | 4 | 14 | 16 | 6 | 12 |
| 25 | 0 | 0 | 0 | 0 | 0 | 0 |

### 5. Hydrophobic Filters and Adsorption

Formulation II comprising compound 1 was prepared as indicated above and filtered using selected sterile filters. Adsorption of compound 1 was determined before and after filtration using different filter types. Table 3 indicates the results of adsorption experiments using different filters.

Only hydrophobic filters show a low amount of compound 1 adsorbed onto the filter material.

TABLE 3

| | Sartorius | Sartorius | Sartorius | |
|---|---|---|---|---|
| | Minisart | Minisart | Minisart | Millipore |
| | High Flow | HY | 0.2 μm | Millex |
| | 0.2 μm | 0.2 μm | (blue) | 0.2 μm |
| Order No | 16532 | 16596 | 16534 | SLGV013SL |
| Filter | PES | PTFE | Celluloseacetat | PVDF |
| membrane | hydrophobic | hydrophobic | hydrophylic | hydrophobic |
| | | | | material that |
| | | | | was render |
| | | | | hydrophilic by |
| | | | | surface |
| | | | | modification. |
| Analysis | 75% | 96.5% | 53% | 97.3% |
| of assay in | | | | |
| filtrate | | | | |

### 6. Adsorption and PTFE Filter

Standard formulation of compound 2 comprising 8.5 mL isotonic saline, 1.5 mL of ethanol and 50 μl sodium phosphate solution.

Table 4 indicates that high amount of the compound 2 is lost during the preparation phase. The bulk comprises the formulation comprising the F18-radiolabeled compound 2 showing a high radioactivity. Radioactivity loss occurs during all steps leading to the Final pharmaceutical formulation ready for administration to patient.

Invention formulation of compound 2 comprising 6.5 mL water for injection, 2 mL of PEG, 1.5 mL of ethanol, 20 mg ascorbic acid and 25 mg sodium phosphate dibasic.

Table 5 indicates that low amount of the compound 2 is lost during the preparation phase. Adsorption is considerably reduced.

TABLE 4

| | | | Decay | |
|---|---|---|---|---|
| | | | correction of | |
| | Radioactivity | | radioactivity to | Percentage |
| | [MBq] | time | EOS [MBq] | [%] |
| Bulk | 1584 | 00:49:02 | | |
| | | (EOS) | | |
| Final | 899.8 | 01:31:25 | 1176 | 74.24 |
| pharmaceutical | | | | |
| formulation | | | | |
| Empty Vial | 16.07 | 01:27:56 | 20.54 | 1.30 |
| containing Bulk | | | | |
| Empty syringe | 19.48 | 01:29:03 | 25.08 | 1.58 |
| used for | | | | |
| transferring | | | | |
| formulation from | | | | |
| bulk to Sterile | | | | |
| filter | | | | |
| Sterile filter | 295.8 | 01:29:52 | 382.8 | 24.17 |

TABLE 5

| | | | Decay | |
|---|---|---|---|---|
| | | | correction of | |
| | Radioactivity | | radioactivity to | Percentage |
| | [MBq] | time | EOS [MBq] | [%] |
| Bulk | 1758 | 17:39:55 | | |
| | | (EOS) | | |
| Final | 1298 | 18:28:00 | 1758 | 100.00 |
| pharmaceutical | | | | |
| formulation | | | | |
| Empty Vial | 14.74 | 18:13:55 | 18.27 | 1.03 |
| containing Bulk | | | | |
| Empty syringe | 4.969 | 18:25:49 | 6.64 | 0.38 |
| used for | | | | |
| transferring | | | | |
| formulation from | | | | |
| bulk to Sterile | | | | |
| filter | | | | |
| Sterile filter | 29.68 | 18:26:52 | 39.92 | 2.27 |

1. A formulation comprising
Lipophilic Amyloid βeta ligand stilbene based derivative,
Alcohol, and
Polyether.

2. The formulation of claim 1 wherein the lipophilic Amyloid βeta ligand stilbene based derivative is a compound of formula I 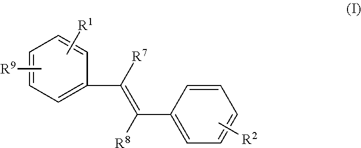
or a suitable salt thereof
wherein
R¹ is selected from the group comprising:
NR³R⁴,
hydroxy,
C₁-C₄ alkoxy,
hydroxy(C₁₋₄)alkyl,
halogen,
cyano,
hydrogen,
nitro,
(C₁-C₄)alkyl,
Halo(C₁-C₄)alkyl, and
Formyl;
R³ and R⁴ are independently hydrogen, C₁-C₄ alkyl or (CH₂)_{d}R⁵, and d is an integer between 1 and 4;
R⁹ is selected from the group comprising R⁵, hydrogen, R⁵—(C₁₋₄)alkyl, [R⁵—(C₁-₄)alkyl]amino, [R⁵—(C₁-C₄)alkyl]alkylamino, and R⁵—(C₁-C₄)alkoxy;
R² is selected from the group consisting of hydroxyl, C₁-₄Alkoxy, (C₁-C₄)-alkyloxo Alk(C₁-C₄)oxy, (C₁-C₄)-alkyloxo(C₁-C₄)-alkyloxo(C₁-C₄)alkoxy, (C₁-C₄)-alkyloxo(C₁-C₄)-alkyloxo(C₁-C₄)-alkyloxo(C₁-C₄)alkoxy, carboxy(C₁-C₄) Alkyl, halo(C₁-C₄)alkoxy, halo(C₁-C₄)-alkyloxo(C₁-C₄)alkoxy, halo(C₁-C₄)alkyloxo(C₁-C₄)alkyloxo-alkyloxy, halo(C₁-C₄)alkyloxo(C₁-C₄)alkyloxo(C₁-C₄)alkyloxo(C₁-C₄)alkyloxy, halo(C₁-C₄)alkyl, NR⁶R¹⁰, phenyl(C₁-C₄)alkyl, R⁵—(C₁-C₄)alkoxy, R⁵—(C₁-C₄)alkyloxo(C₁-C₄)alkoxy, R⁵-(C₁-C₄)alkyloxo(C₁-C₄)alkyloxo(C₁-C₄)alkyloxy, R⁵—(C₁-C₄)alkyloxo(C₁-C₄)alkyloxo(C₁-C₄)alkyloxo(C₁-C₄)alkyloxy, R⁵—(C₁-C₄)alkyl;
R⁵ is ¹⁸F or ¹⁹F;
R⁶ and R¹⁰ are independently selected from the group comprising hydrogen, hydroxy(C₁-C₄)alkyl and C₁-C₄alkyl;
R⁷ and R⁸ are in each instance independently selected from the group comprising halogen, hydrogen, hydroxy, amino, methylamino, dimethylamino, C₁₋₄ alkoxy, C₁₋₄ alkyl, and hydroxy(C₁₋₄)alkyl.

3. The formulation according to claim 1 wherein alcohol is C₂, C₃ or C₄ alcohol in an amount of about 8% v/v to 20% v/v, and polyether is PEG 300, PEG 400 or PEG 1500 in an amount of about 10% v/v to 25% v/v.

4. The formulation according to claim 1 comprising
a compound of formula I 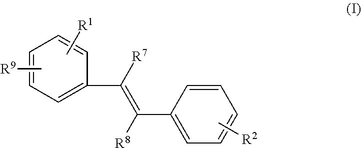
or a suitable salt thereof
wherein
R¹ is NR³R⁴, wherein R³ and R⁴ are independently hydrogen or C₁ alkyl;
R⁹ is hydrogen;
R² is R⁵—C₂-alkyloxo C₂-alkyloxo C₂₋alkyloxy;
R⁷ and R⁹ are hydrogen and
R⁵ is ¹⁹F or ¹⁹F;
Ethanol 96% in an amount of about 10% v/v to 15% v/v and
Polyethylenglycol (PEG 400) in an amount of about 8% v/v to 20% v/v.

5. A method for preparing the formulation according to claim 1.

6. A method for sterile filtration of the formulation according to claim 1 wherein the formulation is given onto a sterile filter, preferably a hydrophobic sterile filter.

7. A method for the preparation of the formulation according to claim 1 comprising a radiotracer obtained through an automated device for radiopharmaceutical use comprising the steps:
Obtaining a radiotracer,
Purification of the radiotracer using a solid-phase-extraction cartridges or column wherein the radiotracer is eluted with alcohol,
Adding the alcohol eluat into polyether for obtaining the invention formulation and
Sterile filtration of the formulation onto a sterile filter;
wherein the radiotracer is a lipophilic Amyloid βeta ligand stilbene based derivative.

8. The formulation according to claim 1 comprising
Compound 1 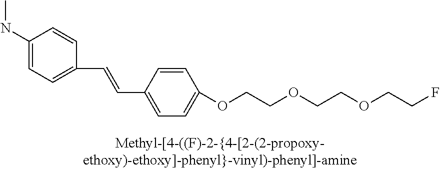
or compound 2 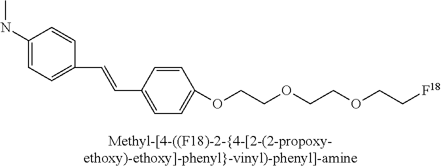
or mixtures thereof
Alcohol,
Polyether, and
pH adjusting agent.
